(19) United States
(12) Patent Application Publication
(10) Pub. No.: US 2025/0009955 A1
(43) Pub. Date: Jan. 9, 2025

(54) STEERABLE SUCTION/IRRIGATION SYSTEM FOR SINGLE-PORT OPERATION

(71) Applicants: Jaschar SHAKURI-RAD, Morgantown, WV (US); Justin R. CHAMBERS, Morgantown, WV (US)

(72) Inventors: Jaschar SHAKURI-RAD, Morgantown, WV (US); Justin R. CHAMBERS, Morgantown, WV (US)

(21) Appl. No.: 18/764,458

(22) Filed: Jul. 5, 2024

Related U.S. Application Data
(60) Provisional application No. 63/525,042, filed on Jul. 5, 2023.

(51) Int. Cl. A61M 1/00; A61B 17/34

(52) U.S. Cl. CPC A61M 1/772; A61B 2017/3425; A61M 2210/1021

(57) ABSTRACT

A medical device includes: a cannula having a distal end and a proximal end opposite the distal end; a housing connected to the proximal end of the cannula; and a manipulator connected to the housing. The cannula includes a rigid section adjacent to the housing and a flexible section adjacent to the distal end of the cannula, and the manipulator is connected to the flexible section in a manner that permits selective bending of the flexible section relative to the rigid section in response to user input at the manipulator. The cannula includes a channel that is configured to convey suction and/or irrigation at the distal end of the cannula.

### PRIORITY

This application claims priority to U.S. provisional patent application No. 63/525,042 filed Jul. 5, 2023, the contents of which are incorporated by reference herein in their entirety.

### FIELD OF THE INVENTION

Aspects of the present invention relate generally to single-port surgery and, more particularly, to devices and methods for providing a steerable suction/irrigation for use in single-port surgery.

### BACKGROUND

Single-port surgery is a procedure performed through one cut (i.e., incision) and with an innovative single-port robot. Surgeons perform single-port surgery through a single port (i.e., incision) in the patient's body, such as in the navel or abdomen. The location of the single incision may vary depending on the surgical procedure. First used clinically in September 2018, single-port robotic surgery is now widely used in many medical centers worldwide for various procedures. Surgeons use this minimally invasive approach in many procedures involving different organ systems. For example, surgeons in a variety of specialties conduct single-port surgery, including urology, and ear, nose and throat. Because it uses only one incision, single-port surgery leaves little to no scarring and may reduce complications that commonly occur after traditional open and even traditional laparoscopic abdominal surgery. People who have single-port surgery report less pain and faster recovery than with traditional minimally invasive surgeries. However, the single-port approach is more challenging than traditional laparoscopy or robotic surgery because your surgeon has less freedom of movement with all instruments using the same entry point.

Current laparoscopic handheld suction/irrigation devices are cumbersome to use and are difficult to maneuver, especially during single-port robotic surgery. Because single-port robotic systems rely on a singular port configuration to reduce the need for additional incisions and ports, this creates space limitations that current tools are not designed to accommodate.

Current tools are also designed with singular functionality and are focused on the suctioning task. In order to manipulate tissue, the suction device has to be taken out of the port and a new tool inserted. This limits the bedside surgeon and assistants.

### SUMMARY

Implementations of the invention address the above-noted problems of the prior art by providing an inventive suction/irrigation device that allows bedside assistants and surgeons to overcome the space limitations faced during laparoscopic/robotic surgery and allow for more efficient surgical procedures. The inventive device allows more efficient and space conscious suctioning, while allowing manipulation of tissue, and allowing specimen retrieval through the port, amongst others. Embodiments described herein include a suction/irrigation device preferably used in single-port laparoscopic operations. A preferred embodiment comprises a suction component, an irrigation component, a cannula, and a cannula tip that is designed to be steerable or manipulable to access hard to reach areas and offer better overall control and manipulation during procedures.

In a first aspect of the invention, there is a medical device comprising: a cannula having a distal end and a proximal end opposite the distal end; a housing connected to the proximal end of the cannula; and a manipulator connected to the housing. The cannula includes a rigid section adjacent to the housing and a flexible section adjacent to the distal end of the cannula, and the manipulator is connected to the flexible section in a manner that permits selective bending of the flexible section relative to the rigid section in response to user input at the manipulator. The cannula includes a channel that is configured to convey suction and/or irrigation at the distal end of the cannula.

In a second aspect, the medical device of the first aspect further comprises: a suction valve; and a suction switch that selectively opens and closes the suction valve to provide suction at the distal end of the cannula.

In a third aspect, the medical device of the first aspect further comprises: an irrigation valve; and an irrigation switch that selectively opens and closes the irrigation valve to provide irrigation at the distal end of the cannula.

In a fourth aspect, the medical device of the first aspect further comprises: a suction valve; a suction switch that selectively opens and closes the suction valve to provide suction at the distal end of the cannula; an irrigation valve; and an irrigation switch that selectively opens and closes the irrigation valve to provide irrigation at the distal end of the cannula.

In a fifth aspect, the medical device of any of the previous aspects further comprises a bend in the cannula along an axial direction of the cannula.

In a sixth aspect, the medical device of any of the previous aspects further comprises a blunt tip at the distal end of the cannula.

In a seventh aspect, the medical device of the sixth aspect further comprises the blunt tip including: a tip channel that conveys suction or irrigation from the channel inside the cannula to an exterior of the blunt tip; and a rounded profile at a distal end of the blunt tip.

In an eighth aspect, the medical device of any of the previous aspects further comprises: a grasping element at the distal end of the cannula; and a grasping actuator that is configured to selectively activate the grasping element in response to user input at the grasping actuator.

In a ninth aspect, the medical device of the eighth aspect further comprises the grasping element including a first part and a second part that is pivotable relative to the first part, wherein the pivoting of the second part relative to the first part is selectively controlled by a user input at the grasping actuator.

In a tenth aspect, the medical device of the eighth aspect further comprises the grasping element including a grasping element channel in fluidic communication with the channel in the cannula, and one or more ports in fluidic communication with the grasping element channel and an exterior of the grasping element, such that the grasping element conveys suction or irrigation from the channel inside the cannula to the exterior of the grasping element.

In an eleventh aspect, the medical device of the eighth aspect further comprises the grasping element being connected to the grasping actuator by a cable that runs in a passageway defined by the cannula.

In a twelfth aspect, the medical device of any of the previous aspects further comprises the manipulator being connected to the flexible section of the cannula by one or more cables that run in one or more passageways defined by the cannula.

In a thirteenth aspect, the medical device of the twelfth aspect further comprises the manipulator being actuatable in only a single direction and is connected to the flexible section via one cable such that the manipulator may be used to bend the flexible section relative to the rigid section in one direction.

In a fourteenth aspect, the medical device of the twelfth aspect further comprises the manipulator being actuatable in two different directions and is connected to the flexible section via two cables such that the manipulator may be used to bend the flexible section relative to the rigid section in two different directions.

In a fifteenth aspect, the medical device of the twelfth aspect further comprises the manipulator being actuatable in three or more different directions and is connected to the flexible section via three or more cables such that the manipulator may be used to bend the flexible section relative to the rigid section in multiple different directions.

In a sixteenth aspect, the medical device of the twelfth aspect further comprises the manipulator being actuatable in four different directions and is connected to the flexible section via four cables such that the manipulator may be used to bend the flexible section relative to the rigid section in four different directions.

In a seventeenth aspect, the medical device of any of the previous aspects further comprises the medical device being configured to be used in single-port surgery.

### BRIEF DESCRIPTION OF THE SEVERAL VIEWS OF THE DRAWINGS

Aspects of the present invention are described in the detailed description which follows, in reference to the noted plurality of drawings by way of non-limiting examples of exemplary embodiments of the present invention.

FIG. 1 shows prior art suction/irrigation device used in single-port robotic surgery.

FIG. 2 shows a suction/irrigation device used in single-port robotic surgery in accordance with aspects of the present disclosure.

FIGS. 3A and 3B show aspects of a suction/irrigation device usable in single-port robotic surgery in accordance with aspects of the present disclosure.

FIGS. 4A and 4B show aspects of a suction/irrigation device usable in single-port robotic surgery in accordance with aspects of the present disclosure.

FIGS. 5A, 5B, and 5C show aspects of a suction/irrigation device usable in single-port robotic surgery in accordance with aspects of the present disclosure.

FIGS. 6A and 6B show aspects of a suction/irrigation device usable in single-port robotic surgery in accordance with aspects of the present disclosure.

FIG. 7 shows aspects of a suction/irrigation device usable in single-port robotic surgery in accordance with aspects of the present disclosure.

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details in more detail than is necessary for the fundamental understanding of aspects of the present invention, the description taken with the drawings making apparent to those skilled in the art how several forms of the present invention may be embodied in practice.

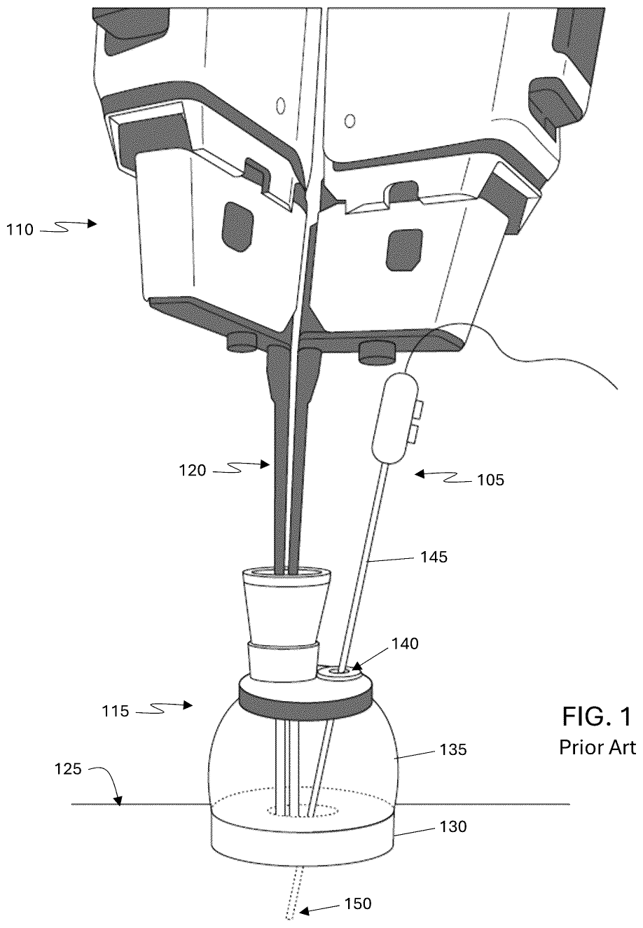

FIG. 1 shows prior art suction/irrigation device 105 used in single-port robotic surgery. In the example shown in FIG. 1, the suction/irrigation device 105 is used with a robotic surgery system such as the DA VINCI SP, which includes a robotic system 110 and an access kit 115. (DA VINCI SP is a trademark of Intuitive Surgical Operations, Inc. of Sunnyvale, California.) The robotic system 110 includes one or more robotic instruments 120 that enter the patient through a single port (i.e., incision) in the skin 125 of the patient. The robotic instruments 120 may include, for example, an endoscope, scissors, cautery instrument, forceps, needle driver, retractor, and clip applier. A surgeon controls movement of the robotic instruments 120 via hand inputs to the robotic system 110. The access kit 115 directly contacts the skin 125 of the patient around the incision, and the robotic instruments 120 access the incision by passing through one or more ports in the access kit 115. The access kit 115 may include a wound protector 130, as well as a clear spherical attachment 135 (also known as a “fishbowl”) which has three separate ports including one for a trocar, one for 12 mm instrumentation, and one for an 8 mm assistant port 140 on the side of the sphere, for example.

With continued reference to FIG. 1, the suction/irrigation device 105 may be used to access the incision via the assistant port 140. The suction/irrigation device 105 includes a rigid tube 145 with a distal end 150, the tube 145 typically constructed of rigid metallic tubing. An operator such as a surgeon or assistant may physically manipulate the position of the suction/irrigation device 105 to attempt to locate the distal end 150 of the suction/irrigation device 105 at a desired location inside the patient. However, when manipulating the position of the suction/irrigation device 105, the suction/irrigation device 105 may come into contact with the structure of the robotic system 110 thereby blocking the ability to locate the distal end of the suction/irrigation device 105 at the desired location inside the patient. Moreover, the straight and rigid tube 145 of the suction/irrigation device 105 combined with the limited range of motion allowed by the combination of the assistant port 140 and incision further makes it difficult to locate the distal end 150 of the suction/irrigation device 105 at the desired location inside the patient. The rigid nature of the tube 145 combined with these space constraints makes it difficult to locate the distal end of the suction/irrigation device 105 at the desired location inside the patient.

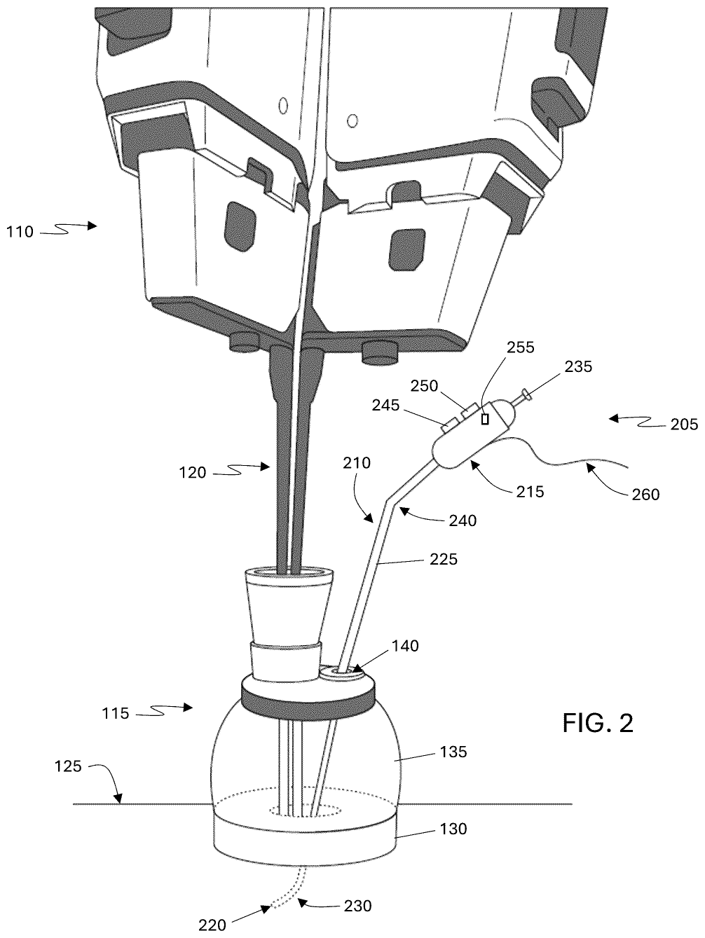

FIG. 2 shows a suction/irrigation device 205 used in single-port robotic surgery in accordance with aspects of the present disclosure. As shown in FIG. 2, the suction/irrigation device 205 may be used with the robotic system 110 of FIG. 1, which includes one or more robotic instruments 120 that access a single incision in a patient skin 125 via an access kit 115. As in FIG. 1, the access kit 115 may include a wound protector 130 and a clear spherical attachment 135, and the robotic instruments 120 and the suction/irrigation device 205 may access the incision via one or more ports (e.g., assistant port 140) formed in the access kit 115.

With continued reference to FIG. 2, in various embodiments the suction/irrigation device 205 includes a cannula 210 and a housing 215. In embodiments, the cannula 210 includes a distal end 220 that is configured to be inserted into the patient, e.g., via the access kit 115 or other type of wound covering device used in single-port surgery. In embodiments, the housing 215 is at a proximal end of the cannula 210 opposite the distal end 220. In accordance with aspects of the present disclosure, the cannula 210 includes a rigid section 225 adjacent to the housing 215 and a flexible section 230 adjacent to the distal end 220. In embodiments and as described herein, the flexible section 230 is manipulatable by an operator via a manipulator 235 of the suction/irrigation device 205 to cause the flexible section 230 to bend in one or more directions relative to the rigid section 225. By way of manipulating the flexible section 230 at the end of the rigid section 225, the location of the distal end 220 of the suction/irrigation device 205 inside the patient may be more precisely controlled compared to using the prior art suction/irrigation device 105 of FIG. 1. In embodiments, the rigid section 225 may include a bend 240 that further assists in positioning the distal end 220 at a desired location inside the patient 125 while avoiding interfering contact between the suction/irrigation device 205 and other structures of the robotic system 110. In this manner, the suction/irrigation device 205 provides an improvement over the prior art suction/irrigation device by overcoming the space constraints that make it difficult to locate the distal end of the prior art suction/irrigation device at a desired location inside the patient, thereby providing for more effective and more efficient surgical procedures compared to the prior art suction/irrigation device.

Still referring to FIG. 2, in embodiments the cannula 210 includes a channel that is in fluid communication with the distal end 220 at one end and suction and irrigation valves that are controlled by a suction switch 245 and irrigation switch 250, respectively, at another end. The suction switch 245 controls (e.g., turns on or off) a suction function of the suction/irrigation device 205 by selectively opening or closing the suction valve. The suction function of the suction/irrigation device 205 provides suction at the distal end 220 via the channel in the cannula 210 and a suction source connected to the suction valve via tubing 260, which may comprise one or more tubes. The irrigation switch 250 controls (e.g., turns on or off) an irrigation function of the suction/irrigation device 205 by selectively opening or closing the irrigation valve. The irrigation function of the suction/irrigation device 205 provides irrigation (e.g., expels liquid, such as water) at the distal end 220 via the channel in the cannula 210 and an irrigation source connected to the irrigation valve via the tubing 260. In this manner, the suction and/or irrigation can be activated and regulated using a valve(s) controlled by a user via switches 245 and 250. The irrigation component may include an activation switch and pump component. The suction and irrigation connections can be made at the back of the housing 215 or anywhere that the design allows for connections of such components. Suction and irrigation tubing, such as tubing 260, may be employed to make these connections at the housing 215.

With continued reference to FIG. 2, in accordance with aspects of the present disclosure, the cannula 210 includes the flexible section 230 adjacent to the distal end 220. The flexible section 230 may include the distal end 220 or may be interposed between the rigid section 225 and a distal end 220 that is also rigid compared to the flexible section 230. The flexible section 230 allows for selectively bending the cannula 210 at the distal end 220 for steerability of the distal end 220. In embodiments, the flexible section 230 comprises a different material or different configuration than the rigid section 225. For example, the rigid section 225 may be composed of rigid plastic or metal, and the flexible section 230 may be composed of silicone or plastic that is less rigid than the material of the rigid section. In one embodiment, the flexible section 230 is more than half the length of the cannula 210 so that the cannula 210 can be selectively bent along a majority of its length. In one embodiment, the rigid section 225 includes a permanent bend 240 relative to an axial direction of the cannula 210, and the flexible section 230 is between the bend 240 and the distal end 220 (with or without including the tip of the distal end 220).

In accordance with aspects of the present disclosure, the manipulator 235 is configured for selectively controlling bending the flexible section 230 relative to the rigid section 225. In various embodiments, the suction/irrigation device 205 includes one or more tension cables connected between the manipulator 235 and the distal end 220. The tension cables may extend through one or more guide channels in or attached to the cannula 210. Movement of the manipulator 235 applies tension force to one or more of the tension cables, which causes the flexible section 230 to bend relative to the rigid section 225. The manipulator 235 may comprise a lever, slide, wheel, joystick, etc., that is used to manipulate the bending of the flexible section 230 by changing the tension of one or more of the tension cables.

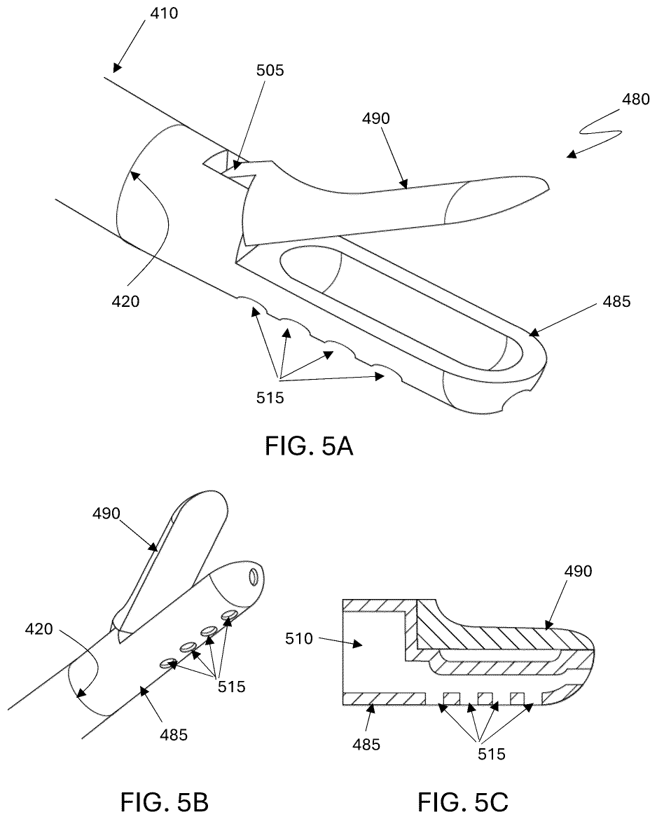

In various embodiments, the suction/irrigation device 205 may further include a grasping actuator 255 that selectively controls (e.g., turns on or off) a grasping function of the suction/irrigation device 205. As discussed herein at FIGS. 5A-C, the distal end 200 may optionally include a grasping component that is selectively actuated by the grasping actuator 255. The grasping actuator 255 may be connected to the grasping component by one or more tension cables that are different than the one or more tension cables associated with the manipulator 235 for bending the flexible section 230. The grasping actuator 255 may comprise a trigger, slide, wheel, or other actuator that is capable of activating a grasping component as described herein. The grasping component provides the suction/irrigation device 205 with a grasping function of for grasping and manipulating tissue inside the patient.

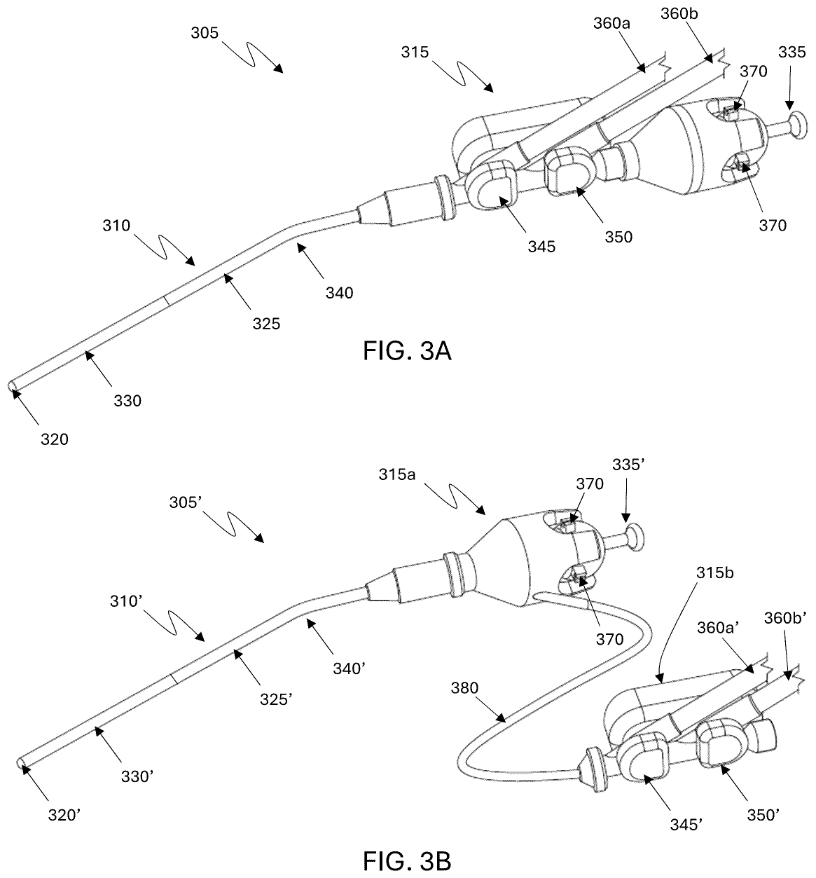

FIG. 3A shows an embodiment of a suction/irrigation device 305 that may be used as the suction/irrigation device 205 of FIG. 2. In the example shown in FIG. 3A, the suction/irrigation device 305 comprises a cannula 310 and housing 315 corresponding, respectively, to the cannula 210 and housing 215 of FIG. 2. The cannula 310 comprises a distal end 320, rigid section 325, flexible section 330, and bend 340 corresponding, respectively, to the distal end 220, rigid section 225, flexible section 230, and bend 240 of FIG. 2. The housing 315 includes a manipulator 335, suction switch 345, and irrigation switch 350 corresponding, respectively, to the manipulator 235, suction switch 245, and irrigation switch 250 of FIG. 2. Tube 360a fluidically connects a suction valve in the housing 315 to a suction source (e.g., vacuum pump) that is external to the device 305, and tube 360b fluidically connects an irrigation valve in the housing 315 to an irrigation source (e.g., water pump) that is external to the device 305. Each of the suction valve and the irrigation valve in the housing 315 are fluidically connected to a channel inside the cannula 310, where the channel extends from the proximal end of the cannula to the distal end 320 of the cannula. A user may selectively apply suction through the cannula, and via an opening at the distal end 320, by actuating the suction switch 345. A user may selectively apply irrigation through the cannula, and via an opening at the distal end 320, by actuating the irrigation switch 350. A user may selectively bend the flexible section 330 relative to the rigid section 325 by manipulating the manipulator 335. In the example shown in FIG. 3A, the manipulator 335 is a joystick that can move in four different directions to control bending of the flexible section 330 relative to the rigid section 325 in four different directions. In the example shown in FIG. 3A, tension wires used in controlling the bending of the flexible section 330 are connected to the manipulator 335 at anchor points 370.

FIG. 3B shows an embodiment of a suction/irrigation device 305′ that may be used as the suction/irrigation device 205 of FIG. 2. In the example shown in FIG. 3B, the suction/irrigation device 305′ comprises a cannula 310′ and housing corresponding, respectively, to the cannula 210 and housing 215 of FIG. 2. The cannula 310′ comprises a distal end 320′, rigid section 325′, flexible section 330′, and bend 340′ corresponding, respectively, to the distal end 220, rigid section 225, flexible section 230, and bend 240 of FIG. 2. The housing includes a first housing section 315a that includes a manipulator 335′ corresponding to the manipulator 235 of FIG. 2. The housing includes a second housing section 315b that includes a suction switch 345′ and irrigation switch 350′ corresponding, respectively, to the suction switch 245 and irrigation switch 250 of FIG. 2. Tube 360a′ fluidically connects a suction valve in the second housing section 315b to a suction source (e.g., vacuum pump) that is external to the device 305′, and tube 360b′ fluidically connects an irrigation valve in the second housing section 315b to an irrigation source (e.g., water pump) that is external to the device 305′. Each of the suction valve and the irrigation valve in the second housing section 315b are fluidically connected to a channel inside the cannula 310′ via another one or more tubes 380 that extend between the first housing section 315a and the second housing section 315b, where the channel extends from the proximal end of the cannula to the distal end 320′ of the cannula. A user may selectively apply suction through the cannula, and via an opening at the distal end 320′, by actuating the suction switch 345′. A user may selectively apply irrigation through the cannula, and via an opening at the distal end 320′, by actuating the irrigation switch 350′. A user may selectively bend the flexible section 330′ relative to the rigid section 325′ by manipulating the manipulator 335′. In the example shown in FIG. 3B, the manipulator 335′ is a joystick that can move in four different directions to control bending of the flexible section 330′ relative to the rigid section 325′ in four different directions. In the example shown in FIG. 3B, tension wires used in controlling the bending of the flexible section 330′ are connected to the manipulator 335′ at anchor points 370′.

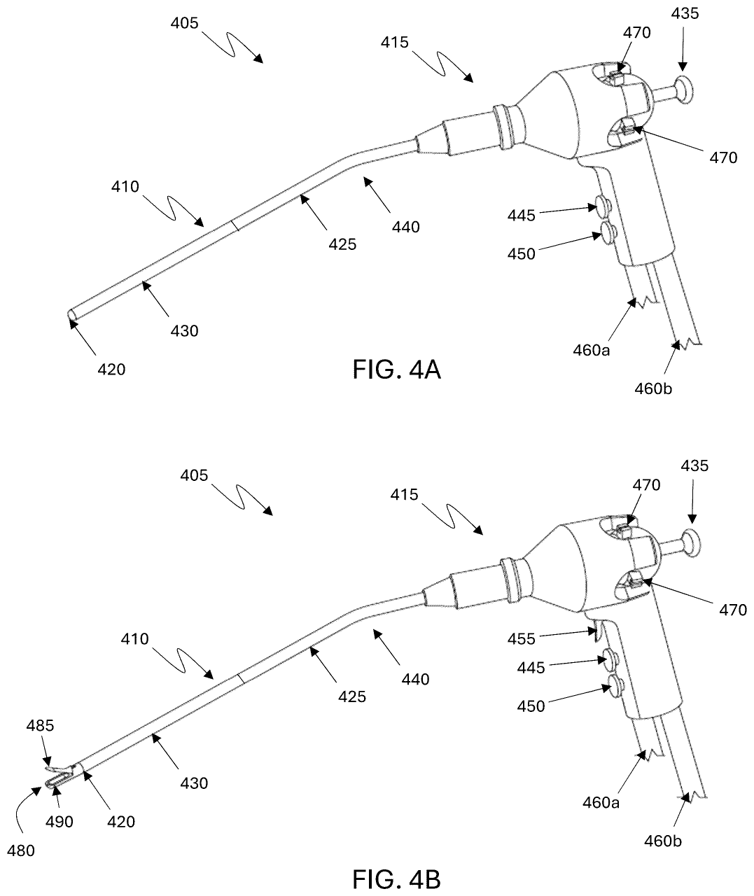

FIG. 4A shows an embodiment of a suction/irrigation device 405 that may be used as the suction/irrigation device 205 of FIG. 2. In the example shown in FIG. 4A, the suction/irrigation device 405 comprises a cannula 410 and housing 415 corresponding, respectively, to the cannula 210 and housing 215 of FIG. 2. The cannula 410 comprises a distal end 420, rigid section 425, flexible section 430, and bend 440 corresponding, respectively, to the distal end 220, rigid section 225, flexible section 230, and bend 240 of FIG. 2. The housing 415 includes a manipulator 435, suction switch 445, and irrigation switch 450 corresponding, respectively, to the manipulator 235, suction switch 245, and irrigation switch 250 of FIG. 2. As illustrated in FIG. 4A, the housing 415 has a pistol-grip shape that differs from the shape of the housing 315 of FIG. 3A. Tube 460a fluidically connects a suction valve in the housing 415 to a suction source (e.g., vacuum pump) that is external to the device 405, and tube 460b fluidically connects an irrigation valve in the housing 415 to an irrigation source (e.g., water pump) that is external to the device 405. Each of the suction valve and the irrigation valve in the housing 415 are fluidically connected to a channel inside the cannula 410, where the channel extends from the proximal end of the cannula to the distal end 420 of the cannula. A user may selectively apply suction through the cannula, and via an opening at the distal end 420, by actuating the suction switch 445. A user may selectively apply irrigation through the cannula, and via an opening at the distal end 420, by actuating the irrigation switch 450. A user may selectively bend the flexible section 430 relative to the rigid section 425 by manipulating the manipulator 435. In the example shown in FIG. 4A, the manipulator 435 is a joystick that can move in multiple (e.g., four) different directions to control bending of the flexible section 430 relative to the rigid section 425 in multiple (e.g., four) different directions. In the example shown in FIG. 4A, tension wires used in controlling the bending of the flexible section 430 are connected to the manipulator 435 at anchor points 470.

FIG. 4B shows an embodiment of a suction/irrigation device 405′ that may be used as the suction/irrigation device 205 of FIG. 2. The suction/irrigation device 405′ of FIG. 4B has many of the same elements as the suction/irrigation device 405 of FIG. 4A, which are represented by the same reference numbers in both figures. The suction, irrigation, and bending of the flexible section 430 operate the same way in the suction/irrigation device 405′ of FIG. 4B as described with respect to the suction/irrigation device 405 of FIG. 4A. In contrast to the suction/irrigation device 405 of FIG. 4A, the suction/irrigation device 405′ of FIG. 4B includes a grasping element 480 at the distal end 420 and a grasping actuator 455 at the housing 415. In embodiments, the grasping element 480 comprises a first part 485 and a second part 490 that is pivotable relative to the first part 485, where the pivoting of the second part 490 relative to the first part 485 is selectively controlled by a user via the grasping actuator 455. For example, pulling the trigger-shaped grasping actuator 455 may cause the second part 490 to rotate toward the first part 485 in a closing motion (e.g., similar to jaws closing), and releasing the trigger-shaped grasping actuator 455 may cause the second part 490 to rotate away the first part 485 in an opening motion (e.g., similar to jaws opening). The grasping element 480 may be provided with one or more holes in fluidic communication with the channel inside the cannula 410 to convey the suction or irrigation through the grasping element 480, e.g., for application to tissue in the patient.

FIGS. 5A, 5B, and 5C show aspects of an exemplary grasping element 480 as shown in FIG. 4B in accordance with aspects of the present disclosure. In embodiments, the grasping element 480 includes a first part 485 and a second part 490 that is pivotable relative to the first part 485. Either of the parts 485 and 490 may be provided with ridges, teeth, roughening, or sharp surfaces to assist in grasping tissue inside the patient. As shown in FIG. 5A, the second part 490 may be pivotally connected to the first part 485 at a hinge 505. The hinge 505 may comprise a pin the defines the pivot axis, a living hinge, or any other suitable type of hinge connection that permits rotational movement of the second part 490 relative to the first part 485. The hinge 505 may include a bias element that urges the second part 490 away from the first part 485, so that the grasping element 480 automatically opens when the user releases the grasping actuator. The bias element may comprise a spring that contacts the second part 490 and the first part 485, or a resiliently flexible material that is integrally formed with one or both of the second part 490 and the first part 485. In embodiments, and as shown in FIGS. 5A and 5B, the grasping element 480 is connected to the cannula 410 at the distal end 420. In embodiments, and as shown in FIGS. 5B and 5C, the grasping element 480 includes a channel 510 that is in fluidic communication with the channel in the cannula 410 when the grasping element 480 is connected to the distal end 420. In embodiments, and as shown in FIGS. 5A-C, the grasping element 480 includes one or more ports 515 in fluidic communication with the channel 510 and the exterior of the grasping element 480, so that the grasping element 480 conveys suction or irrigation from the channel inside the cannula 410 to the exterior of the grasping element 480.

The grasping element 480 shown in FIGS. 5A-C is exemplary and not limiting, and other shapes and configurations of grasping element may be used with the suction/irrigation device 405′ of FIG. 4B. Additionally, although not shown, each of the suction/irrigation device 305 of FIG. 3A and the suction/irrigation device 305′ of FIG. 3B may be modified to include as grasping actuator (e.g., similar to grasping actuator 455 of FIG. 4B) and a grasping element (e.g., similar to grasping element 480 of FIG. 4B).

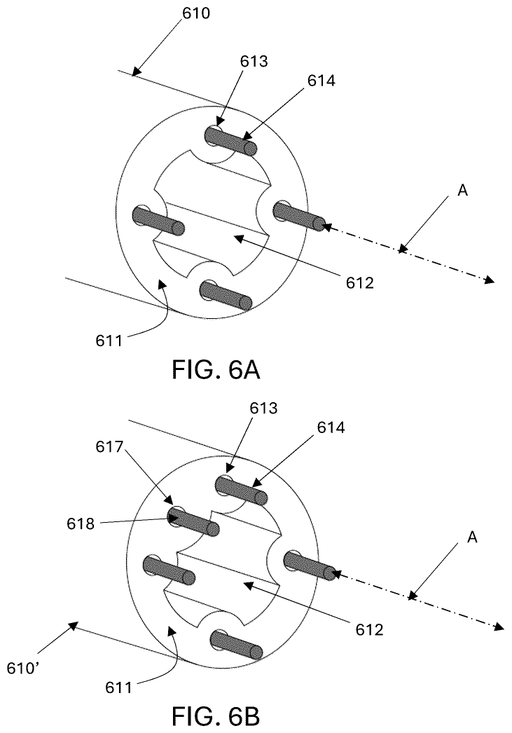

FIGS. 6A and 6B show aspects of a suction/irrigation device usable in single-port robotic surgery in accordance with aspects of the present disclosure. FIG. 6A shows a sectional view of an exemplary cannula 610 that may be used in any of the devices 205, 305, 305′, and 405 as described herein. In embodiments, the cannula 610 includes a cylindrical body 611 (e.g., a tube) that defines the channel 612 that is configured to fluidically communicate suction or irrigation between the respective valves in the housing and the distal end of the cannula, as described herein. In embodiments, and as shown in FIG. 6A, the cannula includes passageways 613 that accommodate tension cables 614. In embodiments, the passageways 613 are separate from and not in fluidic communication with the channel 612. In embodiments, the tension cables 614 may move axially along a length direction of the passageways 613, e.g., as represented by arrow A in FIG. 6A, in response to user input at the manipulator such as manipulator 335 of FIG. 3A. In embodiments, each of the tension cables 614 has one end connected to the manipulator and another end anchored in the flexible section of the cannula.

FIG. 6A shows four passageways 613 and four tension cables 614 (only one set of the four being numbered) for use with a manipulator that is designed to move in four directions, such as manipulator 335 of FIG. 3A. However, implementations may utilize a different manipulator that has a different number of movement directions and is connected to the flexible section via a different number of tension cables. In one example, the manipulator is actuatable in a single direction and is connected to the flexible section via one tension cable such that the manipulator may be used to bend the flexible section relative to the rigid section in one direction. This arrangement provides unidirectional steerability of the cannula tip and allows for steering of the cannula tip in a single direction while rotating the cannula along its axis for additional manipulation. In another example, the manipulator is actuatable in two different directions and is connected to the flexible section via two tension cables, such that the manipulator may be used to bend the flexible section relative to the rigid section in two different directions. This arrangement provides bidirectional steerability in the cannula tip and allows for steering of the cannula tip in two directions while rotating the cannula along its axis for additional manipulation. In another example, the manipulator is actuatable in four different directions and is connected to the flexible section via three or four tension cables, such that the manipulator may be used to bend the flexible section relative to the rigid section in all directions. This arrangement provides multidirectional steerability in the cannula tip and allows for steering of the cannula tip in multiple directions

FIG. 6B shows a sectional view of an exemplary cannula 610′ that may be used in any of the devices described herein that utilize a grasping actuator and grasping element. The cannula 610′ of FIG. 6B includes the cylindrical body 611, channel 612, one or more passageways 613, and one or more tension cables 614 that function in the same manner as described with respect to FIG. 6A. The cannula 610′ of FIG. 6B additionally includes a passageway 617 that accommodates a grasping tension cable 618 that is connected between the grasping actuator (e.g., grasping actuator 455) and grasping element (e.g., grasping element 480). In embodiments, the passageway 617 and grasping tension cable 618 are separate from the one or more passageways 613, and one or more tension cables 614. In embodiments, the grasping tension cable 618 is configured to actuate the grasping element in response to the user providing input at the grasping actuator.

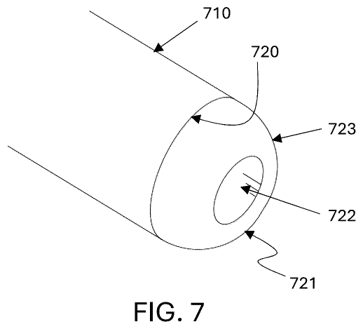

FIG. 7 shows a view of an exemplary cannula 710 that may be used in any of the devices 205, 305, 305′, and 405 as described herein. The cannula 710 includes a distal end 720 which may correspond to the distal ends 220, 320, 320′, and 420. In embodiments, a blunt tip 721 is connected to the distal end 720. In embodiments, the tip 721 includes a channel 722 that is in fluidic communication with the channel in the cannula 710 when the tip 721 is connected to the distal end 720. In this manner, the tip 721 including the channel 722 conveys suction or irrigation from the channel inside the cannula 710 to the exterior of the tip 721. In embodiments, the tip 721 includes a rounded profile 723 at its distal end that reduces trauma to the tissue of the patient. In embodiments, the tip 721 is connected to the distal end 720 or is integrally formed with the distal end 720. In embodiments, a cannula having a tip such as tip 721 may be used when a grasping element is not desired.

Additional aspects of the invention include manufacturing and/or using a suction/irrigation device described herein. Even further aspects of the invention include providing instructions for using a suction/irrigation device described herein. The instructions may be at least one of printed and video.

It is noted that the foregoing examples have been provided merely for the purpose of explanation and are in no way to be construed as limiting of implementations of the present invention. While aspects of the present invention have been described with reference to an exemplary embodiment, it is understood that the words which have been used herein are words of description and illustration, rather than words of limitation. Changes may be made, within the purview of the appended claims, as presently stated and as amended, without departing from the scope and spirit of the present disclosure in its aspects. Although implementations of the present invention have been described herein with reference to particular means, materials and embodiments, implementations of the present invention are not intended to be limited to the particulars disclosed herein; rather, implementations of the present invention extend to all functionally equivalent structures, methods and uses, such as are within the scope of the appended claims.

What is claimed:

1. A medical device, comprising:
a cannula having a distal end and a proximal end opposite the distal end; and
a housing connected to the proximal end of the cannula; and
a manipulator connected to the housing;
wherein the cannula includes a rigid section adjacent to the housing and a flexible section adjacent to the distal end of the cannula, and the manipulator is connected to the flexible section in a manner that permits selective bending of the flexible section relative to the rigid section in response to user input at the manipulator; and
wherein the cannula includes a channel that is configured to convey suction and/or irrigation at the distal end of the cannula.

2. The medical device of claim 1, further comprising:
a suction valve; and
a suction switch that selectively opens and closes the suction valve to provide suction at the distal end of the cannula.

3. The medical device of claim 1, further comprising:
an irrigation valve; and
an irrigation switch that selectively opens and closes the irrigation valve to provide irrigation at the distal end of the cannula.

4. The medical device of claim 1, further comprising:
a suction valve;
a suction switch that selectively opens and closes the suction valve to provide suction at the distal end of the cannula;
an irrigation valve; and
an irrigation switch that selectively opens and closes the irrigation valve to provide irrigation at the distal end of the cannula.

5. The medical device of claim 1, further comprising a bend in the cannula along an axial direction of the cannula.

6. The medical device of claim 1, further comprising a blunt tip at the distal end of the cannula.

7. The medical device of claim 6, wherein the blunt tip comprises:
a tip channel that conveys suction or irrigation from the channel inside the cannula to an exterior of the blunt tip; and
a rounded profile at a distal end of the blunt tip.

8. The medical device of claim 1, further comprising:
a grasping element at the distal end of the cannula; and
a grasping actuator that is configured to selectively activate the grasping element in response to user input at the grasping actuator.

9. The medical device of claim 8, wherein the grasping element comprises a first part and a second part that is pivotable relative to the first part, wherein the pivoting of the second part relative to the first part is selectively controlled by a user input at the grasping actuator.

10. The medical device of claim 8, wherein the grasping element comprises a grasping element channel in fluidic communication with the channel in the cannula, and one or more ports in fluidic communication with the grasping element channel and an exterior of the grasping element, such that the grasping element conveys suction or irrigation from the channel inside the cannula to the exterior of the grasping element.

11. The medical device of claim 8, wherein the grasping element is connected to the grasping actuator by a cable that runs in a passageway defined by the cannula.

12. The medical device of claim 1, wherein the manipulator is connected to the flexible section of the cannula by one or more cables that run in one or more passageways defined by the cannula.

13. The medical device of claim 12, wherein the manipulator is actuatable in only a single direction and is connected to the flexible section via one cable such that the manipulator may be used to bend the flexible section relative to the rigid section in one direction.

14. The medical device of claim 12, wherein the manipulator is actuatable in two different directions and is connected to the flexible section via two cables such that the manipulator may be used to bend the flexible section relative to the rigid section in two different directions.

15. The medical device of claim 12, wherein the manipulator is actuatable in three or more different directions and is connected to the flexible section via three or more cables such that the manipulator may be used to bend the flexible section relative to the rigid section in multiple different directions.

16. The medical device of claim 12, wherein the manipulator is actuatable in four different directions and is connected to the flexible section via four cables such that the manipulator may be used to bend the flexible section relative to the rigid section in four different directions.

17. The medical device of claim 1, wherein the medical device is configured to be used in single-port surgery.
